(19) United States
(12) Patent Application Publication
(10) Pub. No.: US 2017/0311943 A1
(43) Pub. Date: Nov. 2, 2017

(54) TWO-PART ANCHOR WITH ANCHOR INSERTER

(71) Applicant: Smith & Nephew, Inc., Memphis, TN (US)

(72) Inventors: Mark Edwin Housman, North Attleboro, MA (US); Matthew E. Koski, Westford, MA (US); Hemantkumar Patel, Memphis, TN (US); Marc Joseph Balboa, Memphis, TN (US)

(73) Assignee: Smith & Nephew, Inc., Memphis, TN (US)

(21) Appl. No.: 15/526,225

(22) PCT Filed: Dec. 11, 2015
(86) PCT No.: PCT/US2015/065287

§ 371 Date: May 11, 2017

Related U.S. Application Data
(60) Provisional application No. 62/090,689, filed on Dec. 11, 2014.
(60) Provisional application No. 62/090,656, filed on Dec. 11, 2014.

(51) Int. Cl. A61B 17/04

(52) U.S. Cl. CPC A61B 17/0401; A61B 17/0485; A61B 2017/0414; A61B 2017/0409; A61B 2017/0403; A61B 2017/0437; A61B 2017/0412

(57) ABSTRACT

Disclosed herein are embodiments of a no-hole-preparation suture anchor including a tubular anchor body, a tapered tip coupled to the anchor body, an eyelet extending transversely through the anchor, and a plurality of longitudinal ribs extending along at least a portion of the anchor length. The material of the tapered tip is harder than that of the anchor body to facilitate insertion. An anchor inserter for use in combination with the suture anchor includes an elongated, tubular shaft defining a cannulation extending between a proximal end and a distal end. A plurality of tines extends distally from the distal end of the shaft. The anchor includes a plurality of longitudinally extending slots, dimensioned to receive respective ones of the inserter tines. The slots extend along the length of the anchor body and continue into the proximal end of the tubular shaft of the distal tip. The circumferential position of each of the slots is further selected such that the slots are circumferentially adjacent to, but do not intersect, the eyelet.

### BACKGROUND

Anchors are commonly employed in surgical operations in order to secure sutures at desired locations of a patient's anatomy. For example, the anchor is inserted within the tissue of the desired location and frictional sliding resistance between the anchor and the surrounding tissue inhibits movement of the suture anchor, securing the anchor in place. The frictional sliding resistance is largely determined by the normal force (i.e., residual compression) exerted by the tissue upon the surfaces of the anchor and the contact area over which the bone exerts the normal force. Thus, in general, the fixation strength of the anchor inserted within a tissue, a measure of the pull-out force to remove the suture anchor from the tissue, increases with both increasing normal force and contact area.

One class of anchors, commonly referred to as “no-hole-preparation” anchors, are deposited within the tissue without forming a hole prior to deposition of the anchor. Currently developed anchors include a distal tip that pierces the tissue and clears a hole sufficiently large to allow insertion of a “pound-in barbed” or “screw-in threaded” anchor. In each case, the cross-sectional area of the tissue cleared by the anchor is generally larger than the cross-sectional area of the anchor, resulting in gaps between the outer surface of the anchor and the surrounding tissue. These gaps reduce the amount of frictional contact between the anchor and the tissue and decrease the fixation strength provided by the anchor.

Additionally, in recent years, surgeons have been moving towards the use of smaller anchors in surgical repair operations. The use of smaller anchors may be less invasive and allow for more rapid patient healing. With the user of smaller anchors, however, less surface area is available for frictional engagement with the surrounding bone. Thus, lower fixation strength is observed in smaller anchors. In certain repair operations, the fixation strength may be reduced to an unacceptably low level, jeopardizing the stability of a fixation system employing relatively small anchors.

### SUMMARY

Described herein is a no-hole-preparation anchor having improved fixation strength. The anchor includes a tubular anchor body, a tapered tip coupled to the anchor body, an eyelet extending transversely through the anchor, and a plurality of longitudinal ribs extending along at least a portion of the anchor length. One or more of the ribs further extend from the anchor body to terminate in the distal tip. In this configuration, upon insertion of the suture anchor into bone, the anchor displaces bone material immediately ahead of the anchor, advantageously preserving bone laterally adjacent to the ribs. As a result, the contact area and attendant frictional sliding resistance between the anchor and bone is increased, as compared to existing anchors, increasing the fixation strength provided by the anchor to the bone. A distal tip is further adapted for insertion of the anchor within hard tissue, such as bone, without forming a pilot hole in the tissue. For example, the anchor body and distal tip are formed from different materials. Advantageously, the material of the distal tip is harder than that of the anchor body to facilitate insertion.

Also described herein is an anchor inserter for use in combination with the anchor to facilitate insertion of the anchor into bone. The anchor inserter includes an elongated, tubular shaft defining a cannulation extending between a proximal end and a distal end. A plurality of tines extends distally from the distal end of the shaft. The anchor includes a plurality of longitudinally extending slots, dimensioned to receive respective ones of the inserter tines. The slots extend along the length of the anchor body and continue into the proximal end of the tubular shaft of the distal tip. The circumferential position of each of the slots is further selected such that the slots are circumferentially adjacent to, but do not intersect, the eyelet.

In use, a suture is routed through the eyelet, with the free suture limbs extending laterally from the eyelet. The distal end of the inserter shaft is positioned within the anchor body cumulation, with the tines inserted the slots, and distally advanced until the distal end of the tines contacts the proximal end of the tip. Accordingly, the length of the tines are of sufficient length such that, so positioned, the tines are positioned circumferentially adjacent the eyelet, allowing the suture to be routed therethrough without obstruction or impingement by the inserter tines. Subsequently, the anchor is inserted into a bone, distal tip first. Once the anchor is in position within the patient's anatomy, the suture is secured to the anchor. In a knotless embodiment, the inserter may further include a plug positioned within the shaft cannulation. When the inserter is engaged with the anchor, the plug is transferable to the anchor body cumulation (e.g., by a rod positioned within the inserter cannulation, proximal to the plug) until the suture is secured in place between the distal end of the plug and the proximal end of the distal tip. In alternative embodiments, the anchor may be adapted for a knotted engagement with the suture.

The engagement between the distal tip of the anchor and the distal end of the anchor inserter provides a number of benefits. In one aspect, the inserter tines extend both distally and proximally beyond the longitudinal extent of the eyelet, providing mechanical reinforcement to the eyelet during placement of the anchor within a patient's anatomy. In another aspect, a force and moment couple is formed between the inserter shaft and the anchor tip owing to the physical connection there-between. As a result, mechanical loads generated while pounding in the anchor are transferred to the metal tip directly from the inserter, reducing the insertion load carried by the relatively weaker plastic portion of the anchor. This creates a more robust anchor system capable of insertion into much harder media at more extreme angles of attack.

Embodiments of the disclosed anchors include laterally protruding ribs that extend longitudinally along at least a portion of the length of the suture anchor. In further embodiments, the leading distal edge of each of the ribs possesses a tapered “knife-edge” configuration, advantageously allowing the distal end of the anchor to be wedged into the bone. Additionally, the ribs may mitigate the plow-out elect, preserving contact between the ribs and the surrounding bone along nearly the entire length of the anchor. Furthermore, such ribs provide increased surface area, improving fixation strength. Other embodiments include a plurality of other laterally protruding features proximal to the plurality of ribs, such as circumferential ribs, wings, etc. In this manner, the protruding features may further contribute to the fixation achieved by the anchor without removing bone material adjacent to the ribs,

In one embodiment, the anchor of this disclosure includes an elongated anchor body having a proximal end, a distal end, and a longitudinal axis extending between the proximal and distal ends, the anchor body formed from a first material, a tapered tip having a proximal end and a distal end, the tip coupled to the distal end of the anchor body, the tip formed from a second material harder than the first material, and a plurality of longitudinal ribs extending radially outward from an outer surface of the anchor body along at least a portion of a length of the anchor body. At least one the plurality of longitudinal ribs extends between the anchor body to a position within the tapered tip, proximal to the distal terminus. At least a portion of the proximal end of the tip and the distal end of the anchor body abut one another when engaged. The cross-sectional area of the proximal end of the tip and the distal end of the anchor body are approximately equal at said abutment.

Embodiments of the anchor may include one or more of the following, in any combination. In an embodiment, the first material possesses a hardness selected within the range between about 36 Rockwell C to about 700 MPa Brinell. The first material is selected from the group consisting of polyurethanes, polyesters, polyamides, fluoropolymers, polyolefins, polyimides, polyvinyl chloride (PVC) polyethylene (PE), polyethylene glycol (PEG), polystyrene (PS), polyethyl methacrylate (PMMA), polyglycolic acid (PGA), polylactic acid (PLA), polytetrafluoroethylene (PTFE), polyether ether ketone (PEEK). The second material possesses a hardness selected within the range between about 40 Shore D to about 85 Shore D. The second material is selected from the group consisting of stainless steels, titanium, titanium alloys, cobalt-chromium alloy's, platinum alloys, and palladium alloys, carbon-reinforced polyether ether ketone (PEEK), and glass-reinforced PEEK. The suture anchor further includes an eyelet extending through the anchor body transverse to the longitudinal axis. The plurality of longitudinal ribs are not axially aligned with the eyelet. The anchor further includes a pair of longitudinal channels extending proximally from the eyelet to the proximal end of the anchor body. The anchor further includes a suture positioned within the eyelet, wherein one or more suture limbs extend outside of the eyelet, wherein at least one of the suture limbs is positioned within one of the pair of channels. The anchor further includes a plurality of serrations formed about a circumference of respective ones of the plurality of longitudinal ribs. The pair of longitudinal channels are present on opposing sides of the anchor body. The proximal end of the anchor body comprises one or more circumferential ribs positioned proximally with respect to proximal ends of the plurality of longitudinal ribs. An outermost diameter of at least one of the plurality of circumferential ribs is greater than or equal to an outermost radial extent of the plurality of longitudinal ribs. A ratio of a height of each of the plurality of longitudinal ribs to a width of each of the plurality of longitudinal rib is between about 1:4 and about 20:1. A separation angle of spacing between the plurality of longitudinal ribs is between about 7 degrees and about 60 degrees.

In another embodiment, an anchor system is provided. The anchor system includes an anchor having a tubular anchor body extending between a proximal end and a distal end along a longitudinal axis, an eyelet extending through the anchor body transverse to the longitudinal axis, the eyelet in communication with an anchor body cannulation and dimensioned to receive a suture, a tapered anchor tip engaged with the distal end of the anchor body, and a plurality of longitudinal slots, each of the slots having a first portion formed in a surface of the anchor body cannulation and a second portion extending within the anchor tip, wherein the plurality of slots do not intersect the eyelet. The anchor body is formed from a first material and the anchor tip is formed from a second material that is harder than the first material. The anchor system also includes an anchor inserter having an elongated inserter shaft extending between a proximal end and a distal end, and a plurality of tines extending from the distal end of the inserter shaft, each dimensioned for receipt within a respective one of the plurality of slots of the suture anchor. A length of each of the plurality of tines is dimensioned such that, when the tines are inserted within the slots, the tines extend between the anchor body and the anchor tip for contacting the distal tip with the anchor inserter. The tines extend both distally and proximally beyond the longitudinal extent of the eyelet for inhibiting deformation of the eyelet.

Embodiments of the anchor system may include one or more of the following, in any combination. In an embodiment, the anchor system further comprises a plurality of circumferentially spaced ribs extending radially outward from the outer surface of the anchor. Each of the plurality of ribs further extends longitudinally along at least a portion of a length of the anchor body. At least one of the plurality of ribs extends between the anchor body to a position within the distal tip, proximal to a distal terminus. The anchor inserter further includes a cannulation formed within the inserter shaft, a rod positioned within the cannulation, the rod axially moveable with respect to the inserter shaft, and a generally elongate plug positioned within the inserter cannulation, the plug further dimensioned for receipt within the anchor body cannulation. During engagement of the anchor inserter rod with the anchor, distal advancement of the rod urges the plug from the inserter shaft cannulation to the anchor body cannulation. The anchor system further includes a suture routed within the eyelet, wherein distal advancement of the plug into the anchor body cannulation secures the suture to the anchor by compression of the suture between the proximal end of the anchor tip and a distal end of the plug.

### BRIEF DESCRIPTION OF THE DRAWINGS

The foregoing and other objects, features and advantages will be apparent from the following more particular description of the embodiments, as illustrated in the accompanying drawings.

FIGS. 1A-1B are schematic illustrations of a conventional anchor, including only circumferential ribs, inserted into a bone, demonstrating plow-out of surrounding bone material;

FIGS. 2A-2B are schematic illustrations of embodiments of an anchor of the present disclosure including longitudinal ribs and proximal circumferential ribs;

FIG. 3 is a schematic illustration of an embodiments of an anchor wherein the tip is formed of a separate material from the anchor body;

FIG. 4 is a schematic illustration of an embodiments of an anchor including longitudinal ribs and proximal wings;

FIG. 5 is a photograph illustrating an embodiment of an anchor inserter for use with a suture anchor;

FIGS. 6A-6B are schematic illustrations of a distal tip of the anchor inserter of FIG. 5 in perspective view; (A) outer surface; (B) cut-away;

FIGS. 7A-7B are schematic illustrations of the anchor inserter of FIG. 5 inserted within the anchor of FIG. 3 in cut-away side views;

FIG. 8 is a schematic illustration of the anchor inserter of FIG. 5 inserted within the anchor of FIG. 3 in a cut-away, proximal end-on view;

FIGS. 9A-B are schematic illustrations of embodiments of an anchor in cut-away perspective views; and

FIGS. 10A-D are schematic illustrations of embodiments of a suture threader assembly of this disclosure; (A) side view; (B) perspective view; (C) and (D) cut-away top view.

### DETAILED DESCRIPTION

Examples of the anchor and anchor inserter of this disclosure will now be discussed with reference to the figures.

In the description that follows, like components have been given the same reference numerals, regardless of whether they are shown in different examples. To illustrate example(s) in a clear and concise manner, the drawings may not necessarily be to scale and certain features may be shown in somewhat schematic form. Features that are described and/or illustrated with respect to one example may be used in the same way or in a similar way in one or more other examples and/or in combination with or instead of the features of the other examples.

Comprise, include, and/or plural forms of each are open ended and include the listed parts and can include additional parts that are not listed. And/or is open ended and includes one or more of the listed parts and combinations of the listed parts.

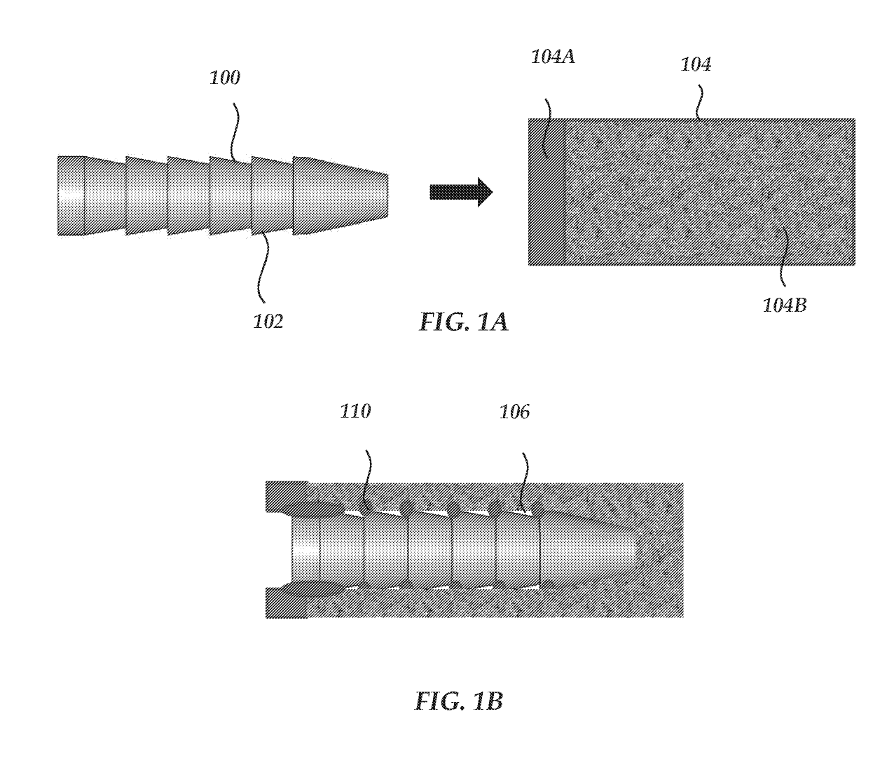

With reference to FIGS. 1A-1B, a conventional suture anchor 100 is illustrated. Typically, in order to enhance the fixation strength, protruding features are often added along the length of the suture anchor 100 (e.g., circumferential ribs 102) to enhance frictional engagement between the suture anchor 100 and the bone 104 upon insertion therein. However, owing to the porous structure of the bone 104, particularly the softer cancellous bone 104B lying beneath the outer cortical bone layer 104A, upon insertion of the anchor 100, the cancellous bone 104B does not deform elastically (i.e., reversibly) to accommodate the anchor 104. Instead, the protruding features 102 remove bone material along their path of insertion, creating void space 106 within their wake between the anchor body and the surrounding bone, referred to as a “plow-out” effect. As a consequence, the amount of bone material in contact with the bone 104 is limited to certain contact points 110 at the outer periphery of the suture anchor 100, rather than the entire surface of the suture anchor 100. Accordingly, the suture anchor 100 may fail to achieve desired levels of fixation, particularly at reduced anchor size.

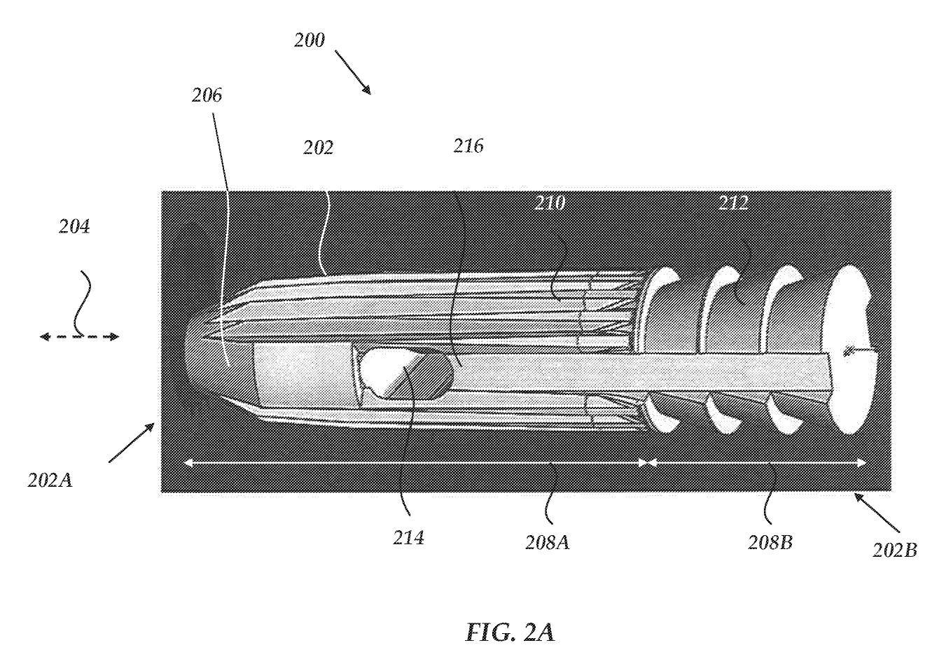

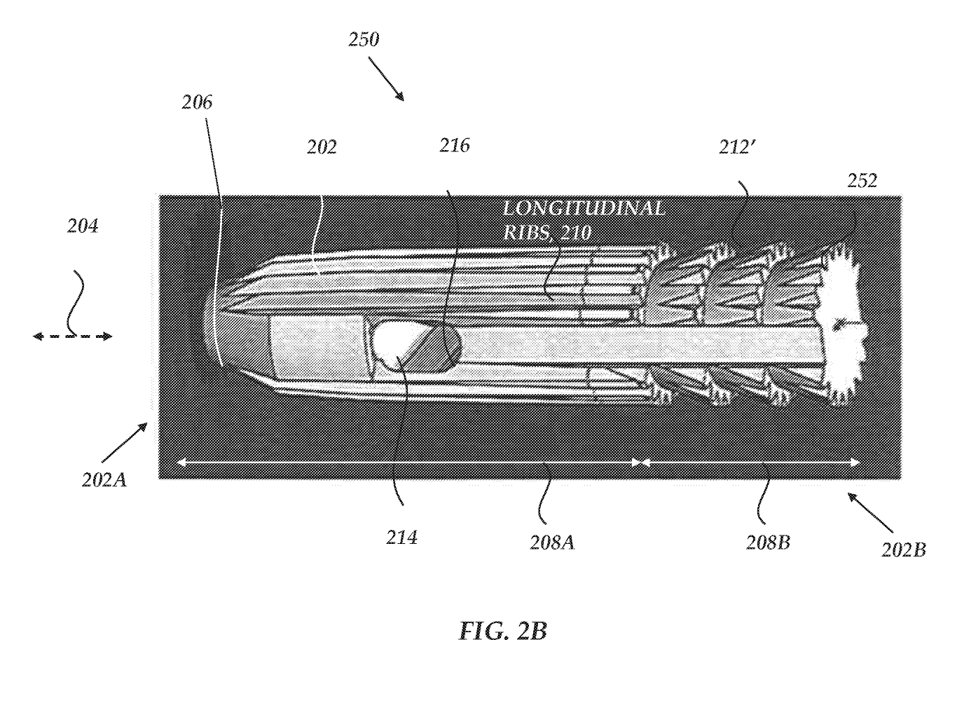

The discussion will now turn to FIGS. 2A-2B, which illustrate embodiments of suture anchors 200, 250 of the present disclosure.

The first suture anchor embodiment 200 is illustrated in FIG. 2A. The suture anchor 200 includes a generally elongated anchor body 202 extending from a distal end 202A to a proximal end 202B along a longitudinal axis 204. The anchor body 202 further includes a distal anchor body section 208A and a proximal anchor body section 208B, as discussed in greater detail below. The proximal end 202B of the anchor 200 is positioned within the proximal anchor body section 208B and is adapted to engage a tool for positioning and insertion of the anchor 200 into a bone. For example, in certain embodiments, the proximal end 202B may include one or more aperture for receipt of a portion of an inserter tool, as further described below. In other embodiments (not shown), the proximal end 202B may be adapted for insertion within an inserter tool.

The distal end 202A of the anchor body 202 is positioned within the distal anchor body section 208A and is further adapted for insertion into bone. For example, as illustrated in FIG. 2A, the distal end 202A of the anchor body 202 includes a tapered distal portion 206. In certain embodiments, the length of the taper 206 ranges between about 10% to about 30% of the total length of the anchor body 202. In other embodiments, the taper may extend along greater portions of the anchor body length, up to and including the entire length. In further embodiments, the tapered distal portion 206 of the anchor body 202 may terminate in a selected geometry. Examples may include, but are not limited to, a generally flat tip (e.g., extending approximately perpendicular to the longitudinal axis), a rounded tip, a sharp tip, and configurations therebetween.

The anchor body 202 further includes a suture eyelet 214. The eyelet 214 extends through the anchor body 202, transverse to the longitudinal axis 204, and is dimensioned to receive a suture. For example, a suture (not shown) may be routed through the eyelet, with free limbs extending adjacent the outer surface of the anchor body. In alternative embodiments, not shown, the eyelet may include a bar, bridge or other protrusion for securing a suture thereto. The suture may be routed through a longitudinal passageway within the anchor body, extending from the proximal end to the eyelet, and secured to the bar or protrusion.

In further embodiments, the anchor 200 includes a plurality of channels 216 formed on the surface of the anchor body 202. For example, as illustrated in FIG. 2A, a pair of channels 216 are present on opposing sides of the anchor 200, extending proximally from the eyelet 214. In use of the anchor 200, the free limbs of a suture routed through the eyelet 214 are positioned within the channels 216. However, it may be understood that, in alternative embodiments, the channels may be omitted from the anchor.

The suture anchor 200 further includes a plurality of longitudinal ribs 210 extending radially outward from, and circumferentially spaced about, the anchor body 202. Each of the plurality of longitudinal ribs 210 extends along at least a portion of the length of the anchor body 202, where a distal end of each longitudinal rib 210 terminates within the tapered distal end 206. For example, as illustrated in FIG. 2A, the longitudinal ribs 210 extend from about the proximal end of the distal anchor body section 208A to within the tapered distal end 206. However, it may be understood that, in alternative embodiments, a proximal end of the longitudinal ribs 210 may be positioned at any location within the distal anchor body section 208A that is proximal to the tapered distal end 206. Furthermore, the distal end of the longitudinal ribs 210 may terminate at any location within the tapered distal end 206. Furthermore, while the each of the longitudinal ribs 210 at a given circumferential position is illustrated as being formed from a single member, in alternative embodiments, a given longitudinal rib 210 may be formed in multiple, discrete segments.

As further illustrated in. FIG. 2A, the longitudinal ribs 210 extend approximately parallel to the longitudinal axis 204. However, in alternative embodiments, at least a portion of the longitudinal ribs 210 may extend at a selected angle with respect to the longitudinal axis 204. It is contemplated by this disclosure that the distal end of the longitudinal ribs 210 may be tapered at an angle greater than that of the anchor body taper. For example, the rib taper angle may be selected within the range between about 25 degrees to about 45 degrees, while the anchor body taper angle may be selected within the range between about 5 degrees to about 25 degrees. A leading edge (e.g., a distally facing edge) of the tapered portion of the longitudinal ribs 210 may include laterally tapered surfaces. This lateral taper, also referred to as a “knife edge” configuration, helps to facilitate insertion of the longitudinal ribs 210 into bone by gradually increasing the surface area of each longitudinal rib 210 in contact with the bone. As a result, the structurally intact bone surrounding the anchor 200 is able to generate a greater reaction force against the surface of the inserted anchor 200. This greater reaction force in turn translates into increased contact pressure which in turn translates into increased anchor fixation strength. Other embodiments of suture anchors having highly tapered ribs are discussed in greater detail in related U.S. application Ser. No. 14/567,400, entitled “Suture anchor Having Improved Fixation Strength” (Atty. Ref. SN-094US), the entirety of which is hereby incorporated by reference.

A height of a respective longitudinal rib 210 is defined by the radial distance that the longitudinal rib 210 extends beyond the anchor body 202. A width of a respective longitudinal rib 210 is given by the average distance between respective lateral sides of the longitudinal rib 210. In certain embodiments, a ratio of rib height to rib width a rib aspect ratio) is selected within the range between about 1:4 and about 20:1. In further embodiments, an anchor core diameter to rib height is selected within the range between about 1:2 to about 1:10. The circumferential spacing of the longitudinal ribs 210 may be varied. For example, a midline of each longitudinal rib 210 is taken as the center point along the rib width. The rib spacing is defined by an angle between adjacent midlines. In certain embodiments, the separation angle is selected between about 7 degrees to about 60 degrees.

As shown in FIG. 2A, the proximal portion 208B of the anchor body 202 may further include a plurality of circumferential ribs 212. Each of the circumferential ribs 212 extends about at least a portion of the circumference of the anchor body 202 at a respective longitudinal position. In an embodiment, the outermost diameter of the plurality of the circumferential ribs 212 is approximately greater than or equal to the outermost radial extent of the longitudinal ribs 210.

In an alternative embodiment, illustrated in FIG. 2B, an anchor 250 is provided. The distal portion 208A of the anchor 250 is the same as anchor 200 of FIG. 2A. However, the proximal portion 208B of the anchor 250 further includes a plurality of serrations 252 formed about the circumference of respective ones of the plurality of circumferential ribs 212 (referred to in the context of anchor 250 as ribs 212′ for clarity). In certain embodiments, the serrations are sawtooth serrations, however other serrations may also be employed without limit. The serrations 252 increase the surface area of the ribs 212′ as compared to the ribs 212, further enhancing fixation of the anchor to bone upon insertion therein.

In use, during advancement of the distal anchor body section 208A of the anchors 200, 250 of FIGS. 2A-B into bone, adjacent bone material is largely preserved. For example, due to the tapered portion of the distal end 206, insertion of the anchors 200, 250 gradually exposes a larger cross-section of the anchors 200, 250, minimizing insertion forces upon the bone and preserving bone integrity. Continued advancement of the distal portion 208A of the anchors 200, 250 proximal to the taper 206 fully inserts the longitudinal ribs 210 into the bone. With the orientation of the longitudinal ribs 210 approximately parallel to the longitudinal axis 204, relatively little bone material adjacent to the longitudinal ribs 210 is removed during insertion of the anchors 200, 250, except that which is directly ahead of the anchors 200, 250. Thus, most of surface area of the ribs 210 is in contact with the bone. Furthermore, the relatively high aspect ratio of the ribs 210 provides greater surface area than would be achieved by the anchor body 202 in their absence, enhancing frictional contact with bone and fixation of the anchor.

With further advancement of the proximal portion 208B of the anchors 200, 250 into the bone, the circumferential ribs 212 are also engaged therewith. While the circumferential ribs 212 plow out some material in their wake, their circumferential extremities still maintain engagement with bone and enhance anchor fixation. For example, when the anchors 200, 250 experience a proximally directed force, the extremities of the circumferential ribs 212 engage the surrounding bone, physically inhibiting proximal retraction of the anchors 200, 250. Thus, the combination of the longitudinal ribs 210 and the circumferential ribs 212 provides enhanced fixation as compared to use of either alone.

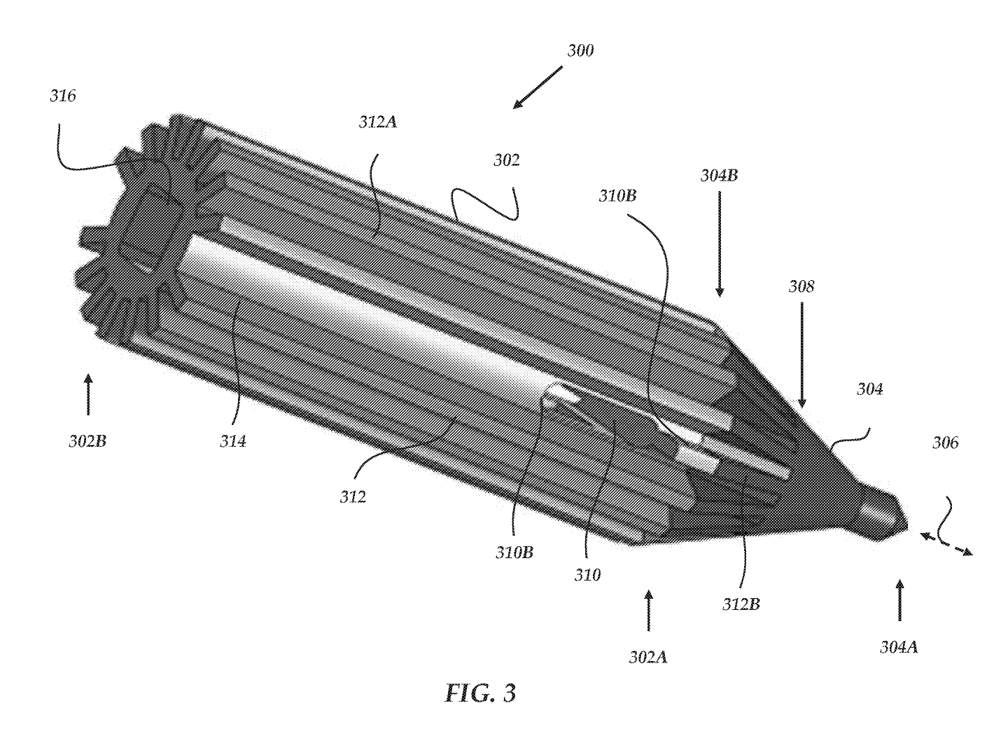

The discussion will now turn to FIG. 3, schematically illustrating another embodiment of an anchor 300. In FIG. 3, the suture anchor 300 includes an anchor body 302 and a tip 304, arranged along a longitudinal axis 306 of the anchor 300. The suture anchor 300 further includes an eyelet 310 and a cannulation 316. The anchor body 302 is generally elongated, extending along the longitudinal axis 306 between a distal end 302A and a proximal end 302B. In certain embodiments, the anchor body 302 is tubular, possessing a circular or elliptical cross-section. In alternative embodiments, the cross-section of the anchor body may adopt different closed shapes. The cannulation 316 extends from the proximal end 302B to the distal end 302A of the anchor body 302 (i.e., the anchor body 302 is fully cannulated). In FIG. 3, the eyelet 310 is shown as being formed through the anchor body 302, transverse to the longitudinal axis 306. However, it is contemplated by this disclosure that the eyelet 310 may be formed through the tip 304. The tip 304 is connected to the distal end 302A of the anchor body 302, as discussed in greater detail below. The tip 304 is generally elongate and tapered, decreasing in cross-sectional area from a tip proximal end 304B to a tip distal end 304A. A taper angle and length of the tip 304 are selected such that the cross-sectional area of the tip proximal end 304B is approximately equal to that of the anchor body distal end 302A. Thus, the cross-sectional area of the anchor as a whole is approximately continuous at the point of abutment between the anchor body 302 and the distal tip 304 (e.g., the distal end 302A of the anchor body 302 and the proximal end 304B of the tip).

In FIG. 3, the anchor 300 further includes a plurality of features extending outwards from the anchor body 302 for engaging bone. For example, the bone engaging features are a plurality of ribs 312 are formed on the outer surface of the anchor 300, circumferentially spaced from one another and extending radially outward there from. The plurality of ribs 312 extend longitudinally along at least a portion of the length of the anchor 300. As further illustrated in FIG. 3, the ribs 312 extend from about the proximal end 302B of the anchor body 302 and terminate within the tapered distal tip 304.

In FIG. 3, the anchor body 302 is further formed from a first material, different from a second material forming the tip 304. Examples of the first material may include, but are not limited to, polyurethanes, polyesters, polyamides, fluoropolymers, polyolefins, polyimides, polyvinyl chloride (PVC) polyethylene (PE), polyethylene glycol (PEG), polystyrene (PS), polymethyl methacrylate (PMMA), polyglycolic acid (PGA), polylactic acid (PLA), polytetrafluoroethylene (PTFE), polyether ether ketone (PEEK). In further embodiments, the anchor body 302 is formed from any material having a hardness within the range between about 36 Rockwell C to about 700 MPA Brinell.

In use, to facilitate displacement of bone upon insertion of the anchor 300 therein, embodiments of the tip 304 are formed from a second material, different from the first material. The second material is harder than the first material, reflecting the fact that the tip 304 is responsible for displacing a majority of the bone volume occupied by the anchor 300, including both the hard, outer cortical bone layer and the underlying cancellous bone (see FIGS. 1A-B). For example, the tip 304 is formed from a material having a hardness within the range between about 40 Shore D to about 85 Shore D. In further embodiments, examples of the second material may include, but are not limited to, stainless steels, titanium, titanium alloys, cobalt-chromium alloys, platinum alloys, and palladium alloys, carbon-reinforced polyether ether ketone (PEEK), and glass-reinforced PEEK.

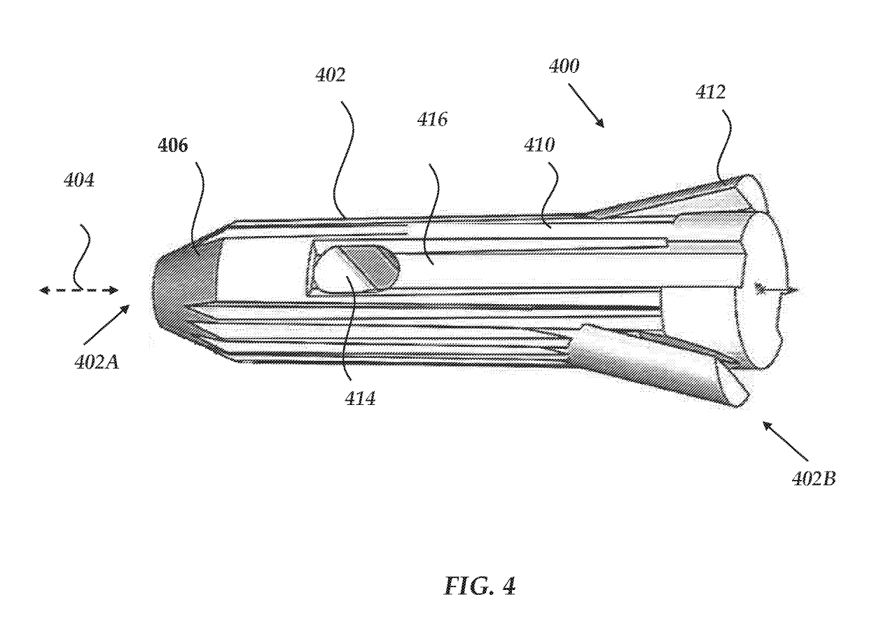

The discussion will now turn to FIG. 4, schematically illustrating another embodiment of an anchor 400. The suture anchor 400 includes a generally elongated anchor body 402 extending from a distal end 402A to a proximal end 402B along a longitudinal axis 404. The proximal end 402B of the anchor 400 is adapted to engage a tool for positioning and insertion of the anchor 400 into a bone, as described further below. The distal end 402A of the anchor body 402 is further adapted for insertion into bone. For example, the distal end 402A of the anchor body 402 includes a taper 406. The anchor body 402 further includes a suture eyelet 414. The eyelet 414 extends through the anchor body 402, transverse to the longitudinal axis 404, and dimensioned to receive a suture. In further embodiments, the anchor 400 includes a plurality of channels 416 formed on the surface of the anchor body 402. The anchor 400 also includes a plurality of longitudinal ribs 410 extending radially outward from, and circumferentially spaced about, the anchor body 402.

The anchor 400 further includes a plurality of wings 412, extending between a distal end and a proximal end. The proximal end of each of the plurality of wings 412 are positioned adjacent to the proximal end 402B of the anchor body 402. The wings 412 are positioned circumferentially such that they do not intersect the plurality of channels 416. The wings are further adapted to move between a closed position, where each wing 412 abuts the anchor body 402 and an open position, where each wing 412 extends outward from the anchor body 402. For example, a distal end of each of the plurality of wings 412 is pivotably attached to the anchor body 402. In an embodiment, the wings 412 are integrally formed with the anchor body 402 and pivot with respect to the anchor body 402 by elastic and/or plastic deformation (i.e., a “live” or “living” hinge). In alternative embodiments, the wings 412 may be separately formed from the anchor body 402 and pivot with respect to the anchor body 402 by rotation about a pin-pivot. The circumferential spacing of the wings 412 may be varied, as necessary. For example, a midline of each wing 412 may be taken as the center point along the wing width. For example, a pair of wings may be separated by an angle of 180 degrees.

In use, during advancement of the anchor 400 into bone, bone material adjacent to the ribs 410 is largely preserved. While the plurality of wings 412 plow out some material in their wake, their circumferential extremities still maintain engagement with bone and augment the fixation provided by the wings 412. For example, after insertion into a bone, the plurality of wings 412 are positioned in the closed position. When experiencing a proximally directed force, the wings 412 move towards the open position and engage the surrounding bone, physically inhibiting proximal retraction of the anchor 400. Thus, the combination of the ribs 410 and wings 412 provides enhanced fixation as compared to use of either alone.

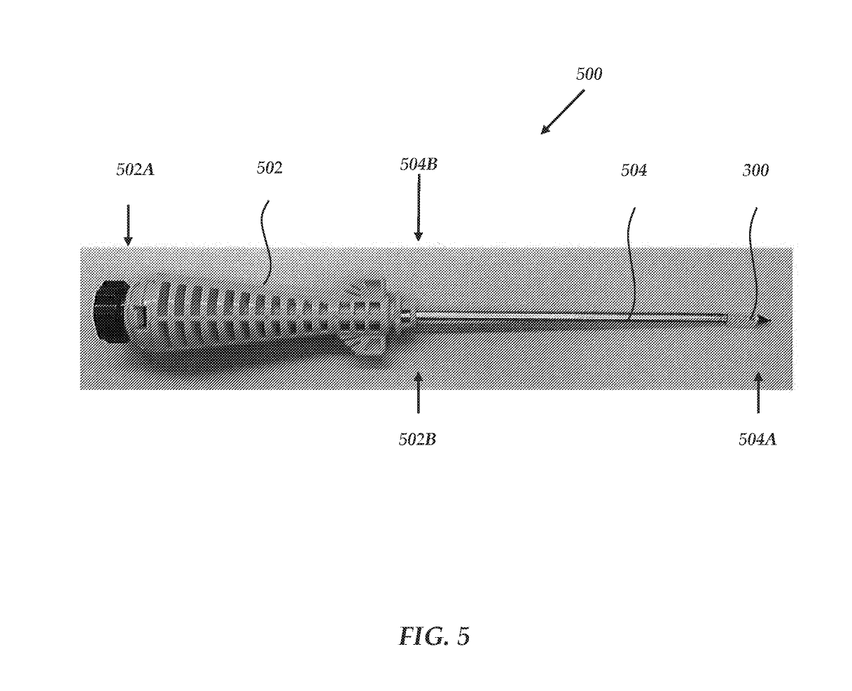

The discussion will now turn to FIG. 5 which presents an embodiment of an anchor inserter 500 for use in combination with embodiments of any of the suture anchors 200, 250, 300 and 400 described above. The inserter 500 includes a handle 502 and a generally elongated inserter shaft 504. The inserter shaft 504 extends between a distal end 504A and a proximal end 504B along a longitudinal axis. The longitudinal axis of the inserter is approximately coincident with the anchor longitudinal axis when embodiments of the anchor are mounted to the distal end 504A of the shaft 504. The proximal end of the inserter shaft 504 is adapted for fixed engagement with a distal end of the handle 502. The distal end the inserter shaft 504A is adapted to engage embodiments of the suture anchor, as discussed below.

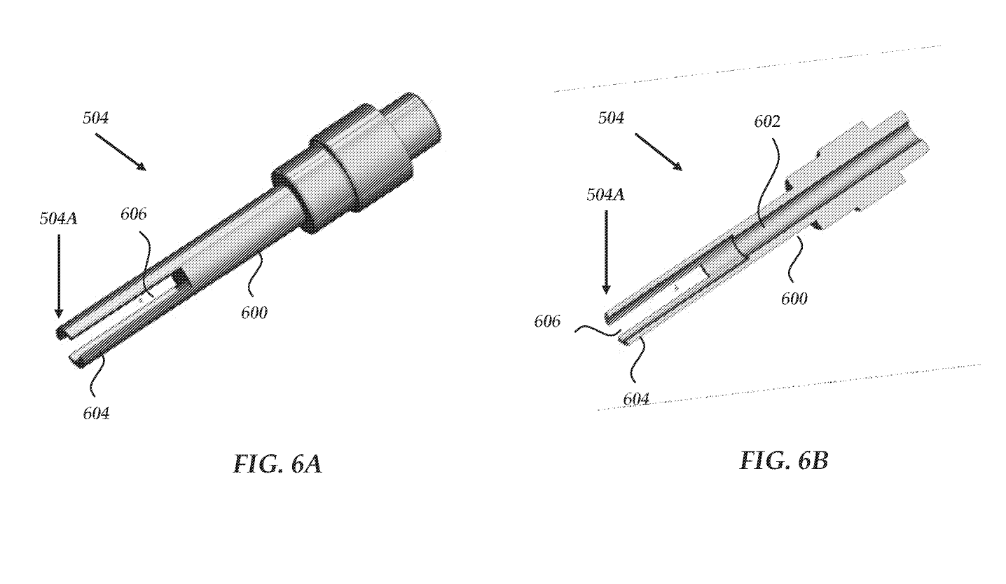

With reference to FIGS. 6A-6B, the distal end 504A of the inserter shaft 504 is illustrated in solid and cut-away views, respectively. The distal end 504A of the inserter shaft 504A includes a shaft body 600 and a cannulation 602 extending therethrough. A plurality of elongate tines 604 are further formed at the distal end of the inserter shaft 504, spaced apart by a through opening 606. As discussed in greater detail below, the plurality of tines 604 are dimensioned for receipt within the anchor via the anchor body cannulation. When the tines 604 are inserted within the anchor body cannulation, the tines 604 do not intersect the eyelet. Thus, the tines 604 do not block the eyelet (or passage of a suture there through) when inserted within the anchor body cannulation. As illustrated in FIGS. 6A-6B, the inserter shaft 504 includes two tines 604. However, in alternative embodiments, the number of tines may be varied, as necessary. The shape of the tines 604 are adapted to mate with the anchor and permit forces and moments to be applied directly to the anchor tip without blocking the eyelet.

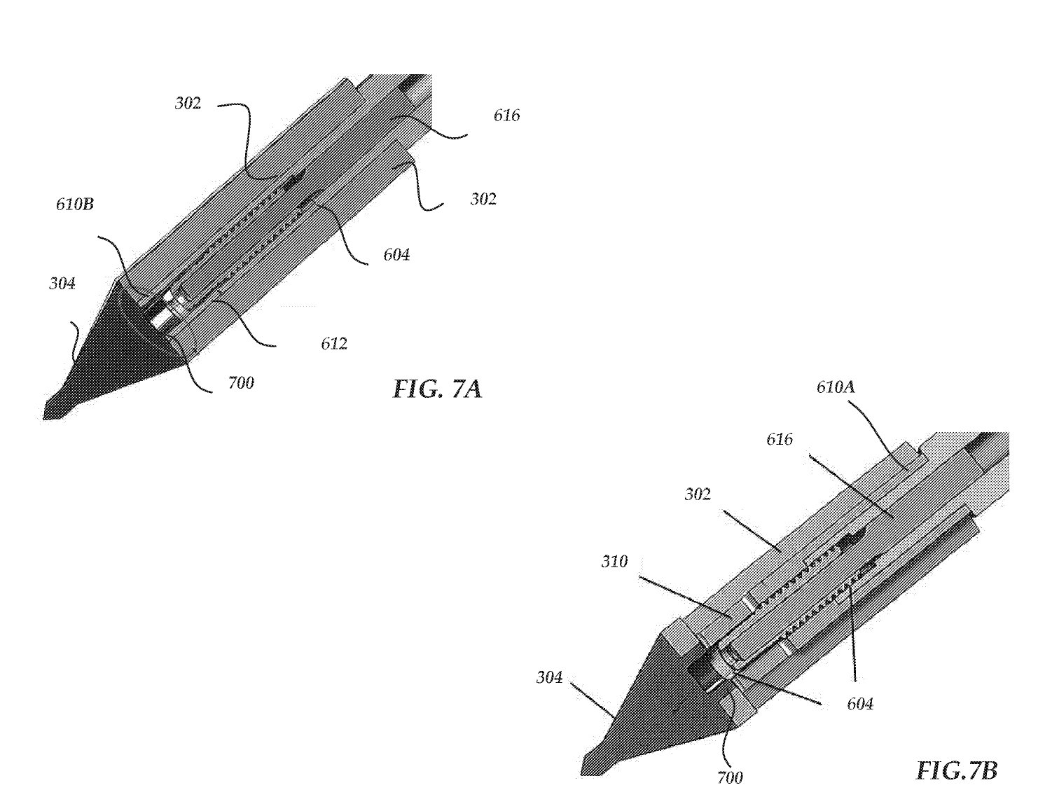

FIGS. 7A and 7B illustrate cross-sectional views of an embodiment of the distal end 504A of the anchor inserter 500 positioned within an anchor. For convenience, the anchor 300 of FIG. 3 is illustrated, although it should be understood that any of the anchors 200, 250, 300 and 400 described above can be used. The views of FIGS. 7A and 7B are at from perpendicular directions, where the eyelet 310 extends through the page in the view of FIG. 7A and the eyelet 310 extends parallel to the page in the view of FIG. 7B. The anchor 300 includes a cut-out region 610A (FIG. 7B) formed on an inner surface of the anchor body cannulation 316. The tip 304 includes a corresponding cut-out region 610B (FIG. 7A). When the tip shaft 700 is inserted within the cannulation 316, the cut-out regions 610A, 610B together define a plurality of slots 612 extending from the proximal end 302B of the anchor body 302 to the distal tip 304. That is to say, in the embodiment of anchor 300, the slots 612 extend distally beyond the eyelet 310 and into the distal tip 304. As illustrated in FIGS. 7A-7B, the slots 612 do not intersect or impinge the eyelet 310.

The plurality of tines 604 are dimensioned for receipt within the slots 612. For example, the cross-sectional area of the tines 604 are approximately the same as, or smaller than, the cross-sectional area of the slots 612. In further embodiments, the length of the tines 604 are dimensioned such that, when inserted within the slots 612, the tines 604 extend between the anchor body 302 and the anchor tip 304 and contact the proximal end of the tip 304 while not intersecting or blocking the eyelet 310. In this manner, a force and moment couple is formed between the inserter shaft 504 and the anchor tip 304 owing to the physical connection there-between. As a result, mechanical loads generated while pounding in the anchor 300 are transferred to the tip 304 directly from the inserter shaft 504, reducing the insertion load carried by the relatively weaker plastic portion of the anchor 300. Furthermore, the tines 604 extend circumferentially adjacent to the eyelet 310 and also distally and proximally beyond the longitudinal extent off the eyelet 310. This arrangement advantageously provides further mechanical reinforcement to the eyelet 310. The combination of these features enhances the mechanical durability of the anchor 300, rendering it capable of insertion into much harder media and/or at more extreme angles of attack.

In further embodiments, the anchor 300 and inserter 500 are further adapted to facilitate knotless engagement of a suture to the anchor 300. For example, the inserter shaft cannulation 602 (FIG. 6B) and anchor body cannulation 316 (FIG. 3) are both dimensioned to receive a generally elongate plug 616 therein. As discussed below, transfer of the plug from the inserter 500 to the anchor 300 secures the suture to the anchor 300. The distal end 504A of the anchor inserter 500 (i.e., the tines 604) is inserted within the slots 612. The anchor inserter 500 is distally advanced within the slots 612 until the tines 604 contact the proximal end of the tip 304. A suture is further routed through the eyelet 310, the free suture limbs extending from the eyelet 310 are tensioned in the proximal direction and positioned within the plurality of channels 314.

The plug 616 is distally advanced from the inserter shaft cannulation 602 into the anchor body cannulation 316 to secure the suture to the anchor. For example, a distal end of the plug 616 is distally advanced into contact with the suture. Further distal advancement of the plug 616 urges the distal end of the plug 616 and the suture into a tip shaft cannulation 700. The suture is secured to the anchor by compression between the proximal end of the tip 304 and the distal end of the plug 616. Beneficially, positioning of the plug 616 within the tip shaft cannulation 700 and anchor body cannulation 316 further provides axial and lateral support to both the tines 604 and the anchor body 302.

After the anchor 300 is mounted to the inserter 500, the anchor 300 is positioned at a desired insertion location with respect to a bone and inserted therein by applying an axial force to the inserter 500, towards the bone. The axial force is applied to the inserter 500 manually (e.g., by hand, or using a tool such as a hammer) or a mechanical mechanism (e.g., a spring loaded or electrically powered impact device, as understood in the art, etc.). The axial force applied to the inserter 500 is transmitted to the anchor 300 primarily through the tines 604 to the tip 304. In certain embodiments, at least a portion of the shaft body 600 proximal to the tines possesses a diameter larger than that of the anchor body cannulation 316 and contacts the proximal end of the anchor body 302. Accordingly, a minority portion of the axial force applied to the device 500 is transmitted to the anchor 300 via impingement of the shaft body 600 proximal to the tines 604 distally against the proximal end of the anchor body 302.

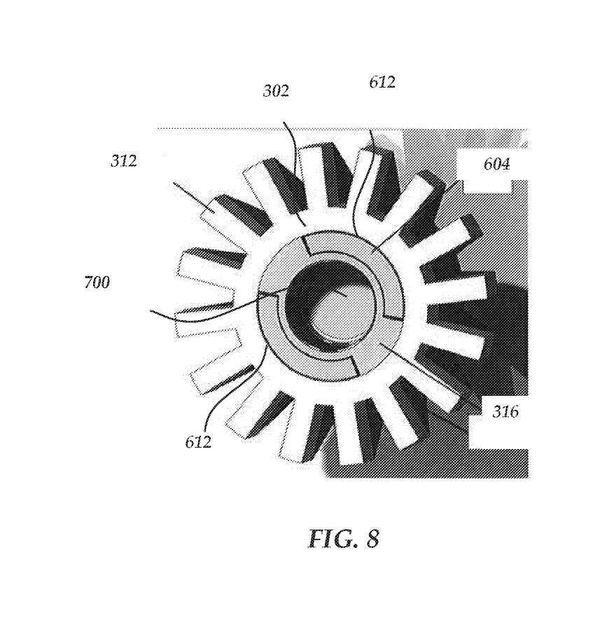

The axial force acts to drive at least a portion of the length of the anchor 300 into the bone. Application of the axial force to the anchor 300 continues until the entire length of the anchor 300 is inserted within the bone. Concurrently, the portions of the suture positioned within the eyelet 310 and channels 314 are drawn into the bone with the anchor 300. The suture is constrained in place with respect to the anchor 300 both by both the plug 616, as well as frictional sliding resistance arising from compression of the suture limbs against the anchor 300 by the surrounding bone. The remainder of the suture limbs extend proximally from the anchor body 302 and are manipulated by a surgeon as necessary for the desired repair operation. FIG. 8 illustrates of the anchor inserter of 500 inserted within the anchor 300 in a cut-away, proximal end-on view.

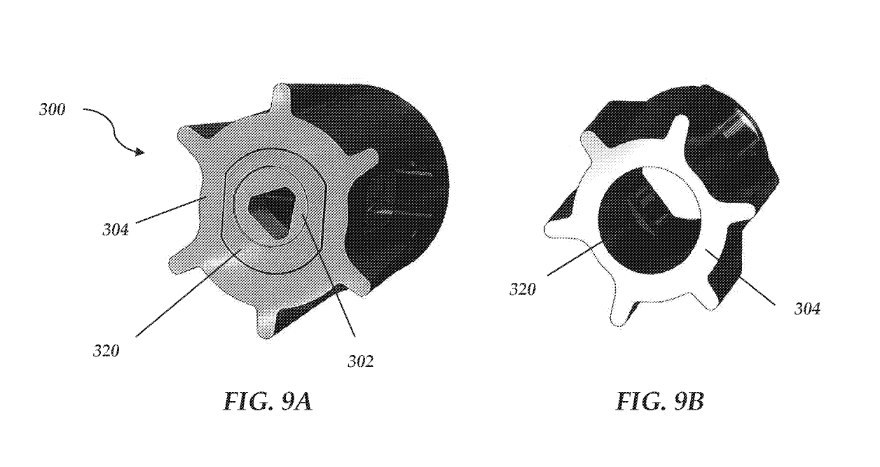

The discussion will now turn to FIGS. 9A and 9B which present an embodiment of an anchor, such as the anchor 300 shown in FIG. 3, with a rotational locking mechanism between a tip 304 formed separately from the anchor body 302 and the anchor body 302. This embodiment is useful when the anchor body 302 and tip 304 each contain eyelets which need to be rotationally aligned with each other to allow a suture to pass therethrough. The embodiments shown in FIGS. 9A and 9B comprise a metal implant tip 304 with an eyelet (not shown) and a polymer anchor body 302 with an eyelet (not shown). The anchor body 302 contains a female socket feature to receive a protruding male feature on the tip 304. Both the anchor body 302 and the tip 304 contain mating projections 320 that serve to rotationally lock the anchor body 302 and tip 304 to each other, thereby aligning the eyelets to one another. Though rotationally locked, the anchor body 302 and tip 304 can slide translationally with respect to each other, advantageously allowing for ease of assembly. FIG. 9A is a cross-sectional view of the anchor 300 showing the interface between the tip 304 and the anchor body 302. As can be seen in FIG. 9A, projections 320 rotationally lock the tip 304 to the anchor body 302 but allow translational motion. FIG. 9B is cross-sectional view from the proximal end of the anchor body 302 with one of the projections 320 visible. The tip 304 and anchor body 302 are connected translationally when in vivo by sutures (not shown) which have been passed through the eyelets. It is contemplated by this disclosure that less than two, or more than two, projections 320 could be used. A shape other than flat (concave, convex, etc.) could also be used. The projections 320 could extend any length, up to the full length of the anchor body 302.

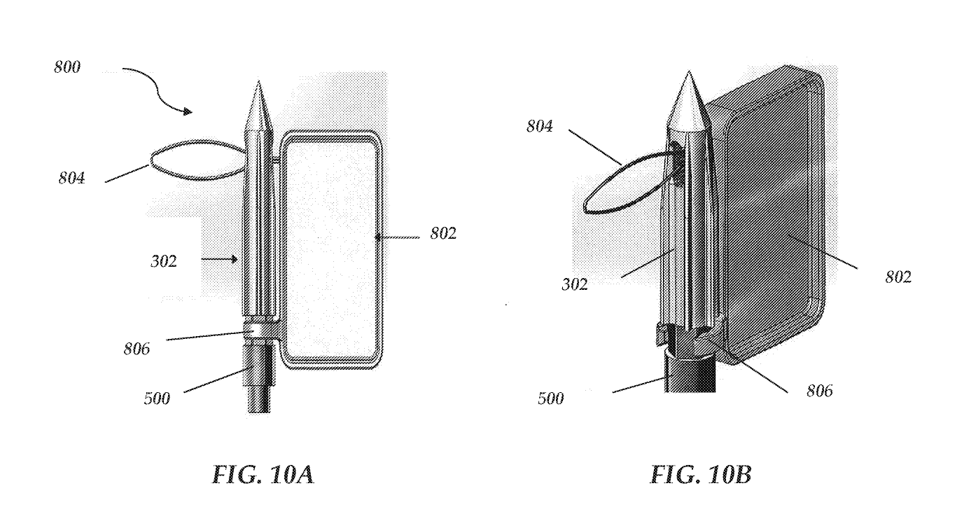

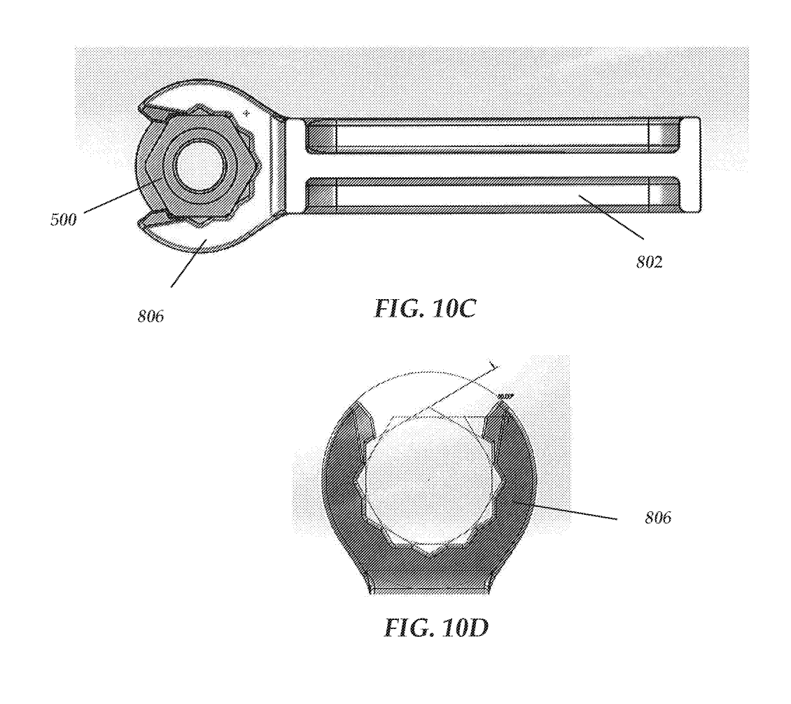

The discussion will now turn to FIGS. 10A-D, which presents embodiments of a suture threader assembly 800 comprising a device 802, such as a tab made from plastic or metal, which can function as a suture threader for threading sutures through an eyelet and also advantageously can include an anti-rotational capability between an anchor and an inserter. This rotation could be a result of vibrations during shipping and handling, or a force inadvertently applied during an operation. Such a rotation reduces the resultant stiffness of the suture threader assembly 800 and can cause failure during anchor insertion.

As shown in FIGS. 10A and 10B, the suture threader assembly 800 includes an anchor, such as the anchor 300 shown in FIG. 3, having an anchor body 302 with a transverse distal eyelet 310. The suture threader assembly 800 also includes an inserter, such as the inserter 500 shown in FIG. 5, partially disposed within the anchor body 302. The device 802 includes a feeder wire 804, which may be comprised of plastic, metal, stainless steel, nitinol or other suitable materials, extending through the eyelet 310. The device 802 also includes a groove or fingers 806 configured to snap over the portion of the inserter 500 extending outside of the anchor body 302. As shown in FIG. 10C, a cross-section of the interface between the groove 806 and the inserter 500 shows that an internal geometry of the groove 806 matches an external geometry of the inserter 500 (shown as a hexagonal geometry), such that the groove 806 can only be snapped onto the inserter 500 in certain positions. This particular arrangement limits rotational movement between the anchor body 302 and inserter 500 in either direction. As shown in FIG. 10D, the groove 806 has a secondary hexagonal cut that allows the device 802 to ratchet into position every 30 degrees. This 30 degree offset allows the anchor body 302 to only rotate 15 degrees in either direction before further motion is resisted. It is contemplated by this disclosure that more than one hexagonal cut could reduce incremental motion of the device 802 relative to the inserter 500. It is further contemplated by this disclosure that a feature protruding through the eyelet would further limit motion. Depending on the clearances, this feature could possibly limit the motion completely. It is further contemplated by this disclosure that the suture threading ability and the anti-rotation ability of the suture threader assembly 800 are provided by separate devices, such as a small clip using a similar geometry at an interface between the feeder wire 804 and the anchor body 302, as well as the interface between the groove 806 and the inserter 500.

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

1. An anchor, comprising:
an elongated anchor body having a proximal end, a distal end, and a longitudinal axis extending between the proximal and distal ends, the anchor body formed from a first material;
a tapered tip having a proximal end and a distal end, the tip coupled to the distal end of the anchor body, the tip formed from a second material harder than the first material; and
a plurality of longitudinal ribs extending radially outward from an outer surface of the anchor body along at least a portion of a length of the anchor body;
wherein at least one the plurality of longitudinal ribs extends between the anchor body to a position within the tapered tip, proximal to a distal terminus; and
wherein at least a portion of the proximal end of the tip and the distal end of the anchor body abut one another when engaged;
wherein the cross-sectional area of the proximal end of the tip and the distal end of the anchor body are approximately equal at said abutment.

2. The anchor of claim 1, wherein the first material possesses a hardness selected within the range between about 36 Rockwell C to about 700 MPa Brinell.

3. The anchor of claim 1, wherein the first material is selected from the group consisting of polyurethanes, polyesters, polyamides, fluoropolymers, polyolefins, polyimides, polyvinyl chloride (PVC) polyethylene (PE), polyethylene glycol (PEG), polystyrene (PS), polymethyl methacrylate (PMMA), polyglycolic acid (PGA), polylactic acid (PLA), polytetrafluoroethylene (PTFE), polyether ether ketone (PEEK).

4. The anchor of claim 1, wherein the second material possesses a hardness selected within the range between about 40 Shore D to about 85 Shore D.

5. The anchor of claim 1, wherein the second material is selected from the group consisting of stainless steels, titanium, titanium alloys, cobalt-chromium alloys, platinum alloys, and palladium alloys, carbon-reinforced polyether ether ketone (PEEK), and glass-reinforced PEEK.

6. The anchor of claim 1, further comprising an eyelet dimensioned to receive a suture extending through the anchor body transverse to the longitudinal axis.

7. The anchor of claim 6, wherein the plurality of longitudinal ribs are not axially aligned with the eyelet.

8. The anchor of claim 6, further comprising a pair of longitudinal channels extending proximally from the eyelet to the proximal end of the anchor body.

9. The anchor of claim 8, further comprising a suture positioned within the eyelet, wherein one or more suture limbs extend outside of the eyelet, wherein at least one of the suture limbs is positioned within one of the pair of channels.

10. The anchor of claim 1, further comprising a plurality of serrations formed about a circumference of respective ones of the plurality of longitudinal ribs.

11. The anchor of claim 8, wherein the pair of longitudinal channels are present on opposing sides of the anchor body.

12. The anchor of claim 1, wherein the proximal end of the anchor body comprises one or more circumferential ribs positioned proximally with respect to proximal ends of the plurality of longitudinal ribs.

13. The anchor of claim 12, wherein an outermost diameter of at least one of the plurality of circumferential ribs is greater than or equal to an outermost radial extent of the plurality of longitudinal ribs.

14. The anchor of claim 1, wherein a ratio of a height of each of the plurality of longitudinal ribs to a width of each of the plurality of longitudinal rib is between about 1:4 and about 20:1.

15. The anchor of claim 1, wherein a separation angle of spacing between the plurality of longitudinal ribs is between about 7 degrees and about 60 degrees.

16. An anchor system, comprising:
an anchor comprising:
a tubular anchor body extending between a proximal end and a distal end along a longitudinal axis, an eyelet extending through the anchor body transverse to the longitudinal axis, the eyelet in communication with an anchor body cannulation and dimensioned to receive a suture;
a tapered anchor tip engaged with the distal end of the anchor body; and
a plurality of longitudinal slots, each of the slots having a first portion formed in a surface of the anchor body cannulation and a second portion extending within the anchor tip, wherein the plurality of slots do not intersect the eyelet;
wherein the anchor body is formed from a first material and the anchor tip is formed from a second material that is harder than the first material; and
an anchor inserter comprising:
an elongated inserter shaft extending between a proximal end and a distal end; and
a plurality of tines extending from the distal end of the inserter shaft, each of the plurality of tines dimensioned for receipt within a respective one of the plurality of slots of the suture anchor;
wherein a length of each of the plurality of tines is dimensioned such that, when the tines are inserted within the slots, the tines extend between the anchor body and the anchor tip for contacting the distal tip with the anchor inserter, and the tines extend both distally and proximally beyond the longitudinal extent of the eyelet for inhibiting deformation of the eyelet.

17. The anchor system of claim 16, wherein the anchor further comprises a plurality of circumferentially spaced ribs extending radially outward from an outer surface of the anchor body, wherein each of the plurality of ribs further extends longitudinally along at least a portion of a length of the anchor body, wherein at least one of the plurality of ribs extends between the anchor body to a position within the distal tip, proximal to a distal terminus.

18. The anchor system of claim 16, wherein the anchor inserter further comprises:
a cannulation formed within the inserter shaft;
a rod positioned within the cannulation, the rod axially moveable with respect to the inserter shaft; and
a generally elongate plug positioned within the inserter cannulation, the plug further dimensioned for receipt within the anchor body cannulation.

19. The anchor system of claim 18, wherein, during engagement of the anchor inserter rod with the anchor body, distal advancement of the rod urges the plug from the inserter shaft cannulation to the anchor body cannulation.

20. The anchor system of claim 19, further comprising a suture routed within the eyelet, wherein distal advancement of the plug into the anchor body cannulation secures the suture to the anchor body by compression of the suture between a proximal end of the anchor tip and a distal end of the plug.
